(19) United States
(12) Patent Application Publication
(10) Pub. No.: US 2006/0211763 A1
(43) Pub. Date: Sep. 21, 2006

(54) Treatment with Statin and Omega-3 Fatty Acids and a Combination Product Thereof

(76) Inventors: Abdel Fawzy, Easton, PA (US); George Bobotas, Tarpos Springs, FL (US); Shobna Chagam, Edison, NJ (US)

Correspondence Address: ARENT FOX PLLC, 1050 CONNECTICUT AVENUE, N.W., SUITE 400, WASHINGTON, DC, 20036, US

(21) Appl. No.: 11/370,178

(22) Filed: Mar. 8, 2006

Related U.S. Application Data
(60) Provisional application No. 60/659,099, filed on Mar. 8, 2005.

(51) Int. Cl. A61K 31/401; A61K 31/36; A61K 31/22; A61K 31/202

(52) U.S. Cl. 514/423; 514/547; 514/548; 514/460; 514/560

(57) ABSTRACT

A pharmaceutical composition in unit dose form, comprising an essentially homogeneous solution comprising a statin essentially dissolved in solvent system comprising natural or synthetic omega-3 fatty acids or pharmaceutically acceptable esters, derivatives, conjugates, precursors or salts thereof, or mixtures thereof, wherein less than 10% of the statin is undissolved in the solvent system.

The present application claims priority from provisional patent application Ser. No. 60/659,099, filed Mar. 8, 2005. The disclosure of the provisional application is hereby incorporated by reference.

### FIELD OF THE INVENTION

The present invention relates to a method utilizing a unit dosage of a combination of statins and omega-3 fatty acids for the treatment of patients with hypertriglyceridemia, hypercholesteremia, coronary heart disease (CHD), heart failure, cardiac arrhythmias, ischemic dementia, hypertension, coagulation related disorders, nephropathy, retinopathy, cognitive disorders, autoimmune diseases, inflammatory diseases, metabolic syndrome, vascular disease, atherosclerotic disease and related conditions, and the treatment and/or prevention and/or reduction of cardiac events and/or cardiovascular events and/or vascular events and/or symptoms. The present invention also relates to a single administration combination product of statin and omega-3 fatty acids.

### BACKGROUND OF THE INVENTION

In humans, cholesterol and triglycerides are part of lipoprotein complexes in the bloodstream, and can be separated via ultracentrifugation into high-density lipoprotein (HDL), intermediate-density lipoprotein (IDL), low-density lipoprotein (LDL) and very-low-density lipoprotein (VLDL) fractions. Cholesterol and triglycerides are synthesized in the liver, incorporated into VLDL, and released into the plasma. High levels of total cholesterol (total-C), LDL-C, and apolipoprotein B (a membrane complex for LDL-C and VLDL-C) promote human atherosclerosis and decreased levels of HDL-C and its transport complex, apolipoprotein A, which are associated with the development of atherosclerosis. Further, cardiovascular morbidity and mortality in humans can vary directly with the level of total-C and LDL-C and inversely with the level of HDL-C. In addition, researchers have found that non-HDL cholesterol is an important indicator of hypertriglyceridemia, vascular disease, artherosclerotic disease and related conditions. In fact, recently non-HDL cholesterol reduction has been specified as a treatment objective in NCEP ATP III.

Agents, such as statins and omega-3 fatty acids, have been used to treat post-myocardial infarction (MI) and adult endogenous hyperlipidemias of hypercholesterolemias and of hypertriglyceridemias, which are generally categorized as “cardiovascular events”.

Statins, which are 3-hydroxy-3-methyl glutaryl coenzyme A (HMG-CoA) reductase inhibitors, have been used to treat hyperlipidemia and arthrosclerosis, for example. Typically, statin monotherapy has been used to treat cholesterol levels, particularly when a patient is not at an acceptable LDL-C level. Statins inhibit the enzyme HMG-CoA reductase, which controls the rate of cholesterol production in the body. Statins lower cholesterol by slowing down the production of cholesterol and by increasing the liver's ability to remove the LDL-cholesterol already in the blood. Accordingly, the major effect of the statins is to lower LDL-cholesterol levels. Statins have been shown to decrease CHD risk by about one-third. However, statins only appear to have a modest effect on the TG-HDL axis.

Marine oils, also commonly referred to as fish oils, are a good source of two omega-3 fatty acids, eicosapentaenoic acid (EPA) and docosahexaenoic acid (DHA), which have been found to regulate lipid metabolism. Omega-3 fatty acids have been found to have beneficial effects on the risk factors for cardiovascular diseases, especially mild hypertension, hypertriglyceridemia and on the coagulation factor VII phospholipid complex activity. Omega-3 fatty acids lower serum triglycerides, increase serum HDL-cholesterol, lower systolic and diastolic blood pressure and the pulse rate, and lower the activity of the blood coagulation factor VII-phospholipid complex. Further, omega-3 fatty acids seem to be well tolerated, without giving rise to any severe side effects.

One such form of omega-3 fatty acid is a concentrate of omega-3, long chain, polyunsaturated fatty acids from fish oil containing DHA and EPA and is sold under the trademark Omacor®. Such a form of omega-3 fatty acid is described, for example, in U.S. Pat. Nos. 5,502,077, 5,656,667 and 5,698,594, each incorporated herein by reference.

Patients with hypercholesteremia or mixed dyslipidemia often present with blood levels of LDL cholesterol greater than 190 mg/dl and triglyceride levels of 200 mg/dl or higher. The use of diet and single-drug therapy does not always decrease LDL cholesterol and triglycerides adequately enough to reach targeted values in patients with mixed dyslipidemia or hypercholesterolemia with or without a concomitant increase in triglycerides. In these patients, a complementary combination therapy of a dyslipidemic agent and omega-3 fatty acids may be desirable.

Studies have examined the effects of fish oil and statin therapy. One study found that fish oil and lovastatin increases plasma LDL cholesterol and VLDL cholesterol. Saify et al., Pakistan J. of Pharm. Sci. (2003) 16(2): 1-8.

Nakamura et al. investigated the effects of purified EPA and statins on patients with hyperlipidemia. Patients having baseline triglyceride levels of 2.07 mmol/l (about 182 mg/dl) and already treated with 5-20 mg/day pravastatin or 5 mg/day simvastatin were additionally treated for 3 months with 900 or 1800 mg/day purified (>90%) EPA ethyl ester. It was reported that combination treatment significantly reduced triglyceride levels, and significantly increased HDL-C levels, as compared to baseline monotherapy. LDL-C levels were not reported. Nakamura et al., Int. J. Clin. Lab Res. 29:22-25 (1999).

Davidson et al. investigated the effects of marine oil and simvastatin in patients with combined hyperlipidemia. Patients having baseline triglyceride levels of 274.7 mg/dl to 336.8 mg/dl were treated for 12 weeks with 10 mg/day simvastatin and placebo, 7.2 g/day marine oil (SuperEPA® 1200) and placebo, or a combination of simvastatin and SuperEPA®. The content of omega-3 fatty acids in 7.2 g of marine oil used in the study was 3.6 g, with an EPA/DHA ratio of 1.5. Combination treatment was shown to significantly increase HDL-C levels, as compared to marine oil alone. In addition, triglyceride and non-HDL-C levels were significantly reduced with combination treatment. However, non-HDL-C levels were reported to be reduced less with combination treatment than with simvastatin alone. Davidson et al., Am J Cardiol (1997) 80: 797-798.

Hong et al. investigated the effects of fish oil and simvastatin in patients with coronary heart disease and mixed dyslipidemia. Patients having baseline triglyceride levels of 292.8 mg/dl or 269.5 mg/dl were initially treated with 10-20 mg/day simvastatin for 6-12 weeks. Thereafter the patients were treated with simvastatin and placebo or simvastatin and 3 g/day fish oil (Meilekang™). Combined treatment significantly reduced triglyceride levels, as compared to baseline and placebo. In addition, combined treatment numerically increased HDL-C levels, and numerically reduced LDL-C levels, as compared to baseline. However, the changes in HDL-C levels and LDL-C levels were not statistically significant. Hong et al., Chin. Med. Sci. J. 19:145-49 (2004).

Contacos et al. investigated the effects of fish oil and pravastatin on patients with mixed hyperlipidemia. Patients having baseline triglyceride levels of 4.6 to 5.5 mmol/l (404 to 483 mg/dl) were initially treated for 6 weeks with 40 mg/day pravastatin, 6 g/day fish oil (Himega™, containing 3 g of omega-3 fatty acids, with an EPA/DHA ratio of 2:1), or placebo. Thereafter, all patients were treated with pravastatin and fish oil for an additional 12 weeks. Initial treatment with pravastatin significantly reduced LDL-C levels. Combined treatment of pravastatin and fish oil also significantly reduced triglyceride and LDL-C levels. However, the addition of fish oil to pravastatin monotherapy resulted in only a numerical increase in LDL-C levels, which was not statistically significant. Treatment with fish oil alone significantly reduced triglyceride levels, but increased LDL-C levels. Combined treatment for this group significantly reduced LDL-C levels, as compared to fish oil alone (but not as compared to baseline). Contacos et al., Arterioscl. Thromb. 13:1755-62 (1993).

Singer investigated the effects of fish oil and fluvastatin on patients with combined hyperlipidemia. Patients having baseline triglyceride levels of 258 mg/dl were initially treated for two months with 40 mg/day fluvastatin, and thereafter were additionally treated for two months with 3 g/day fish oil (18% EPA and 12% DHA). Thereafter, the patients remained on fluvastatin therapy alone for a final two months. Fluvastatin monotherapy was shown to significantly reduce triglyceride and LDL-C levels, and significantly increase HDL-C levels. Combination treatment significantly reduced triglyceride and LDL-C levels and resulted in an additional numerical reduction of triglyceride and LDL-C levels, as compared to fluvastatin alone. Combination treatment numerically increased HDL-C levels, as compared to monotherapy, although the increase in HDL-C levels with combined treatment was not statistically significant. Singer, Prost. Leukotr. Ess. Fatty Acids 72:379-80 (2005).

Liu et al. investigated the effects of fish oil and simvastatin in patients with hyperlipidemia. Patients having baseline triglyceride levels of 1.54 to 1.75 mmol/l (about 136 to 154 mg/dl) were treated for 12 weeks with 10 mg/day simvastatin, 9.2 g/day fish oil (Eskimo-3), or a combination of simvastatin and Eskimo-3. The fish oil contained 18% EPA, 12% DHA, and a total of 38% omega-3 fatty acids. Combined treatment significantly reduced triglyceride and LDL-C levels, and significantly increased HDL-C levels, as compared to baseline, and significantly reduced triglyceride levels as compared to simvastatin alone. Liu et al., Nutrition Research 23 (2003) 1027-1034.

An additional study concluded that the combined treatment of low-dose pravastatin and fish oil after dinner in post-renal transplantation dislipidemia is more effective to change the lipid profile after renal transplantation. Grekas et al., Nephron (2001) 88: 329-333. One article summarizes the combination drug therapies for dyslipidemia, including the combination of statins and 3-7 mg fish oil per day. The study indicates that combination therapy may further augment the reduction of triglyceride, total cholesterol, and apoliprotein E levels, as compared with patients on a statin alone. Alaswad et a., Curr. Atheroscler. Rep. (1999) 1:44-49. In another study, it was found that the combination of dietary fish oil and lovastatin reduces both very low-density lipoprotein (VLDL) and low density lipoprotein (LDL). Huff et al., Arterosclerosis and Thrombosis, 12(8): 901-910 (August 1992).

Additional studies have examined the effects of statins in combination with administration of omega-3 fatty acids and concluded that a diet rich in omega-3 fatty acids increased the cholesterol-lowering effect of simvastatin, counteracted the fasting insulin-elevating effect of simvastatin and did not decrease serum levels of β-carotene and ubiquinol-10. Jula et al., JAMA 287 (5) 598-605 (Feb. 6, 2002). Another study showed an increase in thiobarbituric acid-malondialdehyde complex (TBA-MDA) by using EPA and DHA and statins (e.g., simvastatin) did not affect this result. Grundt et al., Eur. J of Clin. Nutr. (2003) 57: 793-800.

U.S. Pat. No. 6,720,001 discloses a stabilized pharmaceutical oil-in-water emulsion for delivery of a polyfunctional drug having the drug, an aqueous phase, an oil phase and an emulsifier. Statins are claimed among a list of possible polyfunctional drugs, and fish oil is claimed as one of seven optional components for the oil phase. Moreover, U.S. Patent Application Publication No. 2002/0077317 claims compositions of statins and polyunsaturated fatty acids (PUFAs) (EPA and DHA), while U.S. Patent Application Publication No. 2003/0170643 claims a method of treating a patient, by administering a therapeutic which lowers plasma concentrations of apoB and/or an apoB-containing lipoprotein and/or a component of an atherogenic lipoprotein by stimulating post-ER pre-secretory proteolysis (PERPP) using the combination of fish oils with statins, such as pravastatin, lovastatin, simvastatin, atorvastatin, fluvastatin and cerivastatin.

Studies have also investigated the effect of statins and concentrated omega-3 fatty acids, specifically the Omacor® omega-3 acids. For example, Hansen et al. investigated the effect of lovastatin (40 mg/day) in combination with fish oil concentrate (6 g/day Omacor® omega-3 acids) in patients with hypercholesterolemia. Patients having baseline triglyceride levels of 1.66 mmol/l (about 146 mg/dl) were treated with 6 g/day Omacor® for 6 weeks, followed by 40 mg/day lovastatin for an additional 6 weeks, and a combination of both Omacor® and lovastatin for a final 6 weeks. Lovastatin monotherapy resulted in significant increases in HDL-C levels, and significant decreases in triglyceride and LDL-C levels. After combination treatment, triglyceride and LDL-C levels were further significantly decreased. Hansen et al., Arteriosclerosis and Thrombosis 14(2): 223-229 (February 1994).

Nordoy et al. investigated the effect of atorvastatin and omega-3 fatty acids on patients with hyperlipemia. Patients having baseline triglyceride levels of 3.84 mmol/l (about 337 mg/dl) or 4.22 mmol/l (about 371 mg/dl) were treated with 10 mg/day atorvastatin for 5 weeks. Thereafter, for an additional 5 weeks, atorvastatin treatment was supplemented with 2 g/day Omacor® or placebo. Atorvastatin monotherapy, significantly increased HDL-C levels, and triglyceride and LDL-C levels significantly decreased, as compared to baseline. Combination treatment further increased HDL-C levels, as compared to atorvastatin alone. Triglyceride and LDL-C levels numerically further declined slightly with combination treatment, as compared to atorvastatin monotherapy; however, the decrease was insignificant, and the numerical reduction in triglyceride and LDL-C levels was less than with the reduction experienced by the “atorvastatin+placebo” group. The study concluded that the addition of omega-3 fatty acids to statin (e.g., atorvastatin) treatment was an efficient alternative to treating combined hyperlipemia, as the fatty acids further increased HDL-C and reduced systolic blood pressure. Nordoy et al., Nutr. Metab. Cardiovasc. Dis. (2001) 11:7-16.

Salvi et al. investigated the effects of Omacor® and simvastatin on patients with familial hypercholesterolemia. Patients having baseline triglyceride levels of 1.355 mmol/l (about 119 mg/dl) and already treated with 20-40 mg/day simvastatin were additionally treated with 6 g/day Omacor® for 4 weeks. It was shown that combination treatment significantly decreased triglyceride and LDL-C levels after 2 weeks, as compared to baseline monotherapy. Salvi et al., Curr. Ther. Res. 53:717-21 (1993). Yet another study investigated the effects of omega-3 fatty acids (2 g Omacor® omega-3 acids twice a day) for treating subjects with established CHD and type IIb hyperlipidemia who were already taking simvastatin. The study concluded that the Omacor® omega-3 acids was effective in lowering serum triglyceride levels in patients taking simvastatin. Bhatnagar et al., Eur. Heart J Supplements (2001) 4 (Suppl. D): D53-D58.

Chan et al. studied the combined treatment of atorvastatin (40 mg/day) and fish oil (4 Omacor® omega-3 acid capsules orally at night, 4 g/day) on obese, insulin-resistant men with dyslipidemia studied in a fasted state. Patients having baseline triglyceride levels of 1.7 to 2.0 mmol/l (about 150 to 170 mg/dl) were treated for 6 weeks with: 40 mg/day atorvastatin and placebo; 4 g/day Omacor® and placebo; a combination of atorvastatin and Omacor®; or a combination of placebos. Combination treatment significantly decreased triglyceride, non-HDL-C and LDL-C levels, and significantly increased HDL-C, as compared to the placebo group. Chan et al., Diabetes, 51: 2377-2386 (August 2002). An additional paper investigated the effects of atorvastatin (40 mg/day) and fish oil (4 g/day Omacor® omega-3 acids at night) on obese men with dyslipidemia and insulin resistance. The treatment groups received a placebo, atorvastatin, the Omacor® omega-3 acids, or a combination thereof at night. The paper concluded that combination treatment of statins and fish oil may be the optimal approach for correcting dyslipidemia in obese men. Chan et al., Eur. J of Clin. Invest. (2002) 32: 429-436. Another paper investigated the effects of atorvastatin (40 mg/day) and fish oil (4 g/day Omacor® omega-3 acids at night) on plasma high-sensitivity C-reactive protein concentrations in obese individuals with dyslipidemia. The paper concluded that although fish oil supplementation had no effect on plasma hs-CRP, the addition of fish oil to statins may further optimize lipid-regulating effects by enhancing a decrease in plasma triglycerides and increase in HDL-C. Chan et al., Clinical Chemistry (2002) 48(6): 877-883.

Nordoy et al. investigated the effect of omega-3 fatty acids (3.6 g/day via 4 g/day Omacor® omega-3 acids) and simvastatin (20 mg/day) on patients with combined hyperlipemia. The study concluded that supplementation with the fatty acids reduced hemostatic risk factors and significantly reduced postprandial hyperlipemia. Nordoy et al., Arterioscler. Thromb. Vasc. Biol. (2000) 20:259-265.

Nordoy et al. also investigated the efficiency and the safety of treatment with simvastatin and omega-3 fatty acids in patients with hyperlipidemia. Nordoy et al., J. of Internal Medicine, 243:163-170 (1998). Patients having baseline triglyceride levels of 2.76 mmol/l (about 243 mg/dl) or 3.03 mmol/l (about 266 mg/dl) were treated for 5 weeks with 20 mg/day simvastatin or placebo, then all patients were treated for an additional 5 weeks with 20 mg/day simvastatin. Thereafter, patients were additionally treated with 4 g/day Omacor® or placebo, for a further 5 weeks. The administration of omega-3 fatty acids with simvastatin resulted in moderate reductions in serum total cholesterol and reduction in triglycerol levels. HDL-C levels slightly decreased, and LDL-C levels slightly increased, with the addition of Omacor®, as compared to the baseline monotherapy.

Durrington et al. examined the effectiveness, safety, and tolerability of a combination of Omacor® omega-3 acids and simvastatin in patients with established coronary heart disease and persisting hypertriglyceridemia. Patients having an average baseline triglyceride levels >2.3 mmol/l (average patient serum triglyceride level was 4.6 mmol/l), were treated with 10-40 mg/day simvastatin and 2 g/day Omacor® or placebo, for 24 weeks in a double-blind trial, after which both groups were invited to receive Omacor® for a further 24 weeks in an open study. Combination treatment significantly decreased triglyceride levels within 12 weeks, as compared to baseline monotherapy. In particular, the serum triglyceride levels in patients receiving simvastatin and Omacor® omega-3 acids decreased by 20-30%. In addition, the VLDL cholesterol levels in these patients decreased by 30-40%. LDL-C levels significantly decreased, as compared to baseline monotherapy, only after 48 weeks, although there was a numerical (statistically insignificant) decrease at 12 and 24 weeks. Durrington et al., Heart, 85:544-548 (2001).

Many therapeutic substances are sensitive to environmental influences and their active forms are transformed to degradation products which are often less effective than the active forms. Apart from lower efficacy, degradation products may also cause undesirable side effects. Therefore, it is important that a therapeutic substance be as pure as possible when administered; that is, the percent of degradation products and impurities should be minimal.

It is known that certain statins are sensitive to an acidic environment wherein they are degraded to their lactone forms and different isomers. For example, pravastatin, atorvastatin, itavastatin, and fluvastatin are converted to their lactone forms in an acidic environment.

It is also known that statins which are in the lactone form, e.g. lovastatin and simvastatin, are sensitive to alkaline environment wherein they are converted to the acid form. For example, the lactone ring of simvastatin is known to readily hydrolyze in aqueous solutions to form the β-hydroxy acid. Conversion to the hydroxyl acid is rapid in alkaline solutions and is irreversible. Ellison et al., Analytical Profiles of Drug Substances and Excipients, 22:359-388 (1993). Other mechanisms of degradation of statins may take place in an acidic environment, for example, isomerization in case of pravastatin. (Serrajuddin et al., Biopharm. Sci. 80:830- 834 (1991); Kearney et al., Pharm. Res. 10:1461-1465 (1993)).

Therefore, it would be useful to provide a unit dosage of statins and omega-3 fatty acids that does not degrade over time.

PCT Patent Application Publication No. WO 2006/013602 describes a combination, to be administered in unitary form or in coordinated, sequential form, comprising at least one omega-3 fatty acid, optionally esterified or salified, at least one statin, Coenzyme Q10, resveratrol, at least one policosanol, pantethine, selenium, and zinc.

U.S. Patent Application Publication No. 2006/0034815 discloses a pharmaceutical composition comprising an omega-3 oil and one or more salts of a statin, wherein at least about 80 percent of the statin by weight is present as solid particles in heterogeneous suspension. In another embodiment, the publication provides a pharmaceutical composition comprising an omega-3 oil and one or more salts of a statin, wherein up to 15 percent of the amount of statin by weight is in solution while the amount of remaining statin is present in heterogeneous suspension.

### SUMMARY OF THE INVENTION

While prior studies have shown a correlation between omega-3 fatty acids and certain statins in relation to coronary heart disease and other vascular events, there is an unmet need in the art for a combination product of statins and omega-3 fatty acids, for example, in a unit dosage in homogeneous form. There is also an unmet need in the art for a method of administration of a single administration or unit dosage product. There is a further need to provide a homogeneous unit dosage of statins and omega-3 fatty acids that can avoid significant degradation over time.

The present invention meets the unmet needs of the art, as well as others, by providing a pharmaceutical composition, for example, a unit dosage, comprising statins and omega-3 fatty acids, wherein the statins are contained in a homogeneous solution comprising the omega-3 fatty acids. In one aspect of the embodiment, the combination product is used in the treatment of patients with hypertriglyceridemia, hypercholesteremia, coronary heart disease (CHD), heart failure, cardiac arrhythmias, ischemic dementia, hypertension, coagulation related disorders, nephropathy, retinopathy, cognitive disorders, autoimmune diseases, inflammatory diseases, metabolic syndrome, vascular disease, atherosclerotic disease and related conditions, and the treatment and/or prevention and/or reduction of cardiac events and/or cardiovascular events and/or vascular events and/or symptoms.

Yet other embodiments of the present invention include a unit dosage of a statin and omega-3 fatty acids in which at least 90% of the initial amount of statin in the dosage form at an initial measurement time (t₀) is maintained after one month storage at room temperature and 60% relative humidity.

In still other embodiments of the present invention, the unit dose form of a statin and omega-3 fatty acids takes advantage of the unexpected high solubility of statin in the omega-3 acids. The combined administration of statin and the omega-3 acids therefore requires a low amount of solubilizers in order to achieve a homogeneous composition.

In preferred embodiments the pharmaceutical composition comprises Omacor® omega-3 fatty acids, as described in U.S. Pat. Nos. 5,502,077, 5,656,667 and 5,698,594. In other preferred embodiments the pharmaceutical composition comprises omega-3 fatty acids present in a concentration of at least 40% by weight as compared to the total fatty acid content of the composition.

In still other preferred embodiments the omega-3 fatty acids comprise at least 50% by weight of EPA and DHA as compared to the total fatty acid content of the composition, and the EPA and DHA are in a weight ratio of EPA:DHA of from 99:1 to 1:99, preferably from 1:2 to 2:1.

In variations of the present invention, the statin includes, but is not limited to, simvastatin, rosuvastatin, pravastatin, atorvastatin, lovastatin and fluvastatin. In preferred embodiments the statin used in combination with omega-3 fatty acids is simvastatin.

In yet further preferred embodiments of the present invention the triglyceride levels in the serum of a subject prior to the first administration to the subject of the pharmaceutical composition of the invention is about 150 to about 199 mg/dl, or about 200 to about 499 mg/dl, or greater than 499 mg/dl.

The invention also includes the use of an effective amount of a statin and omega-3 fatty acids for the manufacture of a medicament useful for any of the treatment methods indicated herein.

Other features and advantages of the present invention will become apparent to those skilled in the art upon examination of the following or upon learning by practice of the invention.

### DESCRIPTION OF THE PREFERRED EMBODIMENTS

The present invention is directed to the utilization of statins and omega-3 fatty acids for the treatment of patients with hypertriglyceridemia, hypercholesteremia, coronary heart disease (CHD), heart failure, cardiac arrhythmias, ischemic dementia, hypertension, coagulation related disorders, nephropathy, retinopathy, cognitive disorders, autoimmune diseases, inflammatory diseases, metabolic syndrome, vascular disease, atherosclerotic disease and related conditions, and the treatment and/or prevention and/or reduction of cardiac events and/or cardiovascular events and/or vascular events and/or symptoms, and a combination product or unit dosage comprising one or more statins and one or more omega-3 fatty acids. Preferably, the one or more statins and the one or more omega-3 fatty acids are the only active agents in the combination product.

The present invention may incorporate now known or future known statins in an amount generally recognized as safe. There are currently six statins that are widely available: atorvastatin, rosuvastatin, fluvastatin, lovastatin, pravastatin, and simvastatin. A seventh statin, cerivastatin, has been removed from the U.S. market at the time of this writing. However, it is conceivable to one skilled in the art that cerivastatin may be used in conjunction with some embodiments of the present invention if cerivastatin is ultimately determined to be safe and effective.

Generally, the effect of statins is dose dependent, i.e., the higher the dose, the greater the therapeutic affect. However, the effect of each statin is different, and therefore the level of therapeutic effect of one statin cannot be necessarily be directly correlated to the level of therapeutic effects of other statins. For example, bioavailability varies widely among the statins. Specifically, it has been shown that simvastatin is less than 5% bioavailable, while fluvastatin is approximately 24% bioavailable. Statins are absorbed at rates ranging from about 30% with lovastatin to 98% with fluvastatin. First-pass metabolism occurs in all statins except pravastatin. Pravastatin is also the least protein-bound of the statins (about 50%), compared with the others, which are more than 90% protein-bound. Accordingly, the statins possess distinct properties from one another. The combination products of this invention involving each statin or a plurality of statins are also distinct. Such combinations have not been shown in the prior art.

As used herein, the term “omega-3 fatty acids” includes natural or synthetic omega-3 fatty acids, or pharmaceutically acceptable esters, derivatives, conjugates (see, e.g., Zaloga et al., U.S. Patent Application Publication No. 2004/0254357, and Horrobin et al., U.S. Pat. No. 6,245,811, each hereby incorporated by reference), precursors or salts thereof and mixtures thereof. Examples of omega-3 fatty acid oils include but are not limited to omega-3 polyunsaturated, long-chain fatty acids such as a eicosapentaenoic acid (EPA), docosahexaenoic acid (DHA), and α-linolenic acid; esters of omega-3 fatty acids with glycerol such as mono-, di- and triglycerides; and esters of the omega-3 fatty acids and a primary, secondary or tertiary alcohol such as fatty acid methyl esters and fatty acid ethyl esters. Preferred omega-3 fatty acid oils are long-chain fatty acids such as EPA or DHA, triglycerides thereof, ethyl esters thereof and mixtures thereof. The omega-3 fatty acids or their esters, derivatives, conjugates, precursors, salts and mixtures thereof can be used either in their pure form or as a component of an oil such as fish oil, preferably purified fish oil concentrates. Commercial examples of omega-3 fatty acids suitable for use in the invention include Incromega F2250, F2628, E2251, F2573, TG2162, TG2779, TG2928, TG3525 and E5015 (Croda International PLC, Yorkshire, England), and EPAX6000FA, EPAX5000TG, EPAX4510TG, EPAX2050TG, K85TG, K85EE, K80EE and EPAX7010EE (Pronova Biocare a.s., 1327 Lysaker, Norway).

Preferred compositions include omega-3 fatty acids as recited in U.S. Pat. Nos. 5,502,077, 5,656,667 and 5,698,694, which are hereby incorporated herein by reference in their entireties.

Another preferred composition includes omega-3 fatty acids present in a concentration of at least 40% by weight, preferably at least 50% by weight, more preferably at least 60% by weight, still more preferably at least 70% by weight, most preferably at least 80% by weight, or even at least 90% by weight. Preferably, the omega-3 fatty acids comprise at least 50% by weight of EPA and DHA, more preferably at least 60% by weight, still more preferably at least 70% by weight, most preferably at least 80%, such as about 84% by weight. Preferably the omega-3 fatty acids comprise about 5 to about 100% by weight, more preferably about 25 to about 75% by weight, still more preferably about 40 to about 55% by weight, and most preferably about 46% by weight of EPA. Preferably the omega-3 fatty acids comprise about 5 to about 100% by weight, more preferably about 25 to about 75% by weight, still more preferably about 30 to about 60% by weight, and most preferably about 38% by weight of DHA. All percentages above are by weight as compared to the total fatty acid content in the composition, unless otherwise indicated. Methods of determining the weight percentages are taught, e.g., in U.S. Pat. Nos. 5,502,077, 5,656,667 and 5,698,694. The determination of percentage by weight may be based, e.g., on the ethyl ester form of the omega-3 fatty acids, even if other forms are present.

The EPA:DHA ratio may be from 99:1 to 1:99, preferably 4:1 to 1:4, more preferably 3:1 to 1:3, most preferably 2:1 to 1:2. The omega-3 fatty acids may comprise pure EPA or pure DHA.

The omega-3 fatty acid composition optionally includes chemical antioxidants, such as alpha tocopherol, oils, such as soybean oil and partially hydrogenated vegetable oil, and lubricants such as fractionated coconut oil, lecithin and a mixture of the same.

The most preferred form of omega-3 fatty acids is the Omacor® omega-3 acid (K85EE, Pronova Biocare A.S., Lysaker, Norway) and preferably comprises the following characteristics (per dosage form):

| Test | Minimum Value | Maximum Value |
|---|---|---|
| Eicosapentaenoic acid C20:5 | 430 mg/g | 495 mg/g |
| Docosahexaenoic acid C22:6 | 347 mg/g | 403 mg/g |
| EPA and DHA | 800 mg/g | 880 mg/g |
| Total n-3 fatty acids | 90% (w/w) | |

The combination product of a statin and omega-3 fatty acids may be administered in a capsule, a tablet, a powder that can be dispersed in a beverage, or another solid oral dosage form, a liquid, a soft gel capsule or other convenient dosage form such as oral liquid in a capsule, as known in the art. In some embodiments, the capsule comprises a hard gelatin. The combination product may also be contained in a liquid suitable for injection or infusion.

The active ingredients of the present invention may also be administered with a combination of one or more non-active pharmaceutical ingredients (also known generally herein as “excipients”). Non-active ingredients, for example, serve to solubilize, suspend, thicken, dilute, emulsify, stabilize, preserve, protect, color, flavor, and fashion the active ingredients into an applicable and efficacious preparation that is safe, convenient, and otherwise acceptable for use. Thus, the non-active ingredients may include colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide and xanthum gum.

Surfactants which may be used include glycerol acetates and acetylated glycerol fatty acid esters. Preferred glycerol acetates include acetin, diacetin, triacetin and mixtures thereof. Preferred acetylated glycerol fatty acid esters include acetylated monoglycerides, acetylated diglycerides and mixtures thereof.

In addition, the surfactant may be a glycerol fatty acid ester. The fatty acid component is about 6-22 carbon atoms. The glycerol fatty acid ester can be a monoglyceride, diglyceride, triglyceride or mixtures thereof. Preferred glycerol fatty acid esters include monoglycerides, diglycerides, medium chain triglycerides with fatty acids having about 6-12 carbons and mixtures thereof. Capmul® MCM (medium chain mono- and diglycerides) is an example.

The surfactant may be a propylene glycol ester. Preferred propylene glycol esters include propylene carbonate, propylene glycol monoacetate, propylene glycol diacetate, propylene glycol fatty acid esters, acetylated propylene glycol fatty acid esters and mixtures thereof. Alternatively, the propylene glycol fatty acid ester may be a propylene glycol fatty acid monoester, propylene glycol fatty acid diester or mixture thereof. The fatty acid has about 6-22 carbon atoms. Preferred propylene glycol ester are propylene glycol monocaprylate (Capryol®), propylene glycol dicaprylate, propylene glycol dicaprate, propylene glycol dicaprylate/dicaprate and mixtures thereof.

Another group of surfactants are ethylene glycol esters. Ethylene glycol esters include monoethylene glycol monoacetates, diethylene glycol esters, polyethylene glycol esters and mixtures thereof. Additional examples include ethylene glycol monoacetates, ethylene glycol diacetates, ethylene glycol fatty acid monoesters, ethylene glycol fatty acid diesters, and mixtures thereof. Alternatively, the ethylene glycol ester may be a polyethylene glycol fatty acid monoesters, polyethylene glycol fatty acid diesters or mixtures thereof. Again, the fatty acid component will contain about 6-22 carbon atoms. Particularly preferred ethylene glycol esters are those obtained from the transesterification of polyethylene glycol with a triglyceride or a vegetable oil or mixture thereof and include, for example, those marketed under the Labrafil® and Labrasol® names.

Polyoxyethylene-sorbitan-fatty acid esters (also called polysorbates), e.g. of from 4 to 25 alkylene moieties, for example mono- and tri-lauryl, palmityl, stearyl and oleyl esters of the type known and commercially available under the trade name Tween® are also suitable as surfactants.

One group of preferred surfactants include propylene glycol monocaprylate, mixtures of glycerol and polyethylene glycol esters of long fatty acids, polyethoxylated castor oils, nonylphenol ethoxylates (Tergitol®), glycerol esters, oleoyl macrogol glycerides, propylene glycol monolaurate, propylene glycol dicaprylate/dicaprate, polyethylene-polypropylene glycol copolymer, and polyoxyethylene sorbitan monooleate.

Hydrophilic solvents which may be used include an alcohol, e.g. a water miscible alcohol, e.g. absolute ethanol, or glycerol. Other alcohols include glycols, e.g. any glycol obtainable from an oxide such as ethylene oxide, e.g. 1,2-propylene glycol. Other examples are polyols, e.g. a polyalkylene glycol, e.g. poly(C2-3)alkylene glycol. A typical example is a polyethylene glycol. Alternatively the hydrophilic component may preferably comprise an N-alkylpyrolidone, e.g. N-(C1-14alkyl)pyrolidone, e.g. N-methylpyrolidone, tri(C1-4alkyl)citrate, e.g. triethylcitrate, dimethylisosorbide, (C5-C13)alkanoic acid, e.g. caprylic acid or propylene carbonate.

The hydrophilic solvent may comprise a main or sole component, e.g. an alcohol, e.g. C1-4-alcohol, e.g. ethanol, or alternatively a co-component, e.g. which may be selected from partial lower ethers or lower alkanols. Preferred partial ethers are, for example, Transcutol® (which has the formula C2H5-[O-(CH2)2]2-OH), Glycofurol® (also known as tetrahydrofurfuryl alcohol polyethylene glycol ether), or lower alkanols such as ethanol.

The combination product of a statin and concentrated omega-3 fatty acids is aided by the solubility of the statin in the omega-3 fatty acid oil. In the combination product, the statin is substantially dissolved in the omega-3 fatty acid oil to provide a substantially homogeneous composition. Thus, the combination product does not require high amounts of solubilizers, such as surfactants, hydrophilic or hydrophobic solvents, oils or combinations thereof, to dissolve the statin. Preferably, the statin is contained in the pharmaceutical composition without the use of large amounts of solubilizers (other than the omega-3 fatty acid oil), and is substantially dissolved (i.e., less than 10%, preferably less than 5% remains undissolved in the solvent system). In a preferred embodiment, the statin is completely dissolved. In preferred embodiments, if present at all, solubilizers other than the omega-3 fatty acid oil are present in amounts of 50% or less w/w based on the total weight of the solvent system in the dosage form, preferably 40% or less, more preferably 30% or less, even more preferably 20% or less, still more preferably 10% or less and most preferably 5% or less. In some embodiments, the solvent system contains no solubilizers other than the omega-3 fatty acid oil. As used herein, “solvent system” includes the omega-3 fatty acid oil. In other preferred embodiments, the weight ratio of omega-3 fatty acid oil to other solubilizer is at least 0.5 to 1, more preferably at least 1 to 1, even more preferably at least 5 to 1, and most preferably at least 10 to 1.

In some embodiments, if present at all, the amount of surfactant or hydrophilic solvent, or combinations thereof, used in the solvent system is 20% or less w/w based on the total weight of the solvent system in the dosage form, more preferably 10% or less, and most preferably 5% or less. In certain embodiments, the amount of surfactant or hydrophilic solvent, or combinations thereof, used in the solvent system is between 1 and 10% w/w, preferably between 2 and 8% w/w. It is preferred that two or more of surfactant(s), hydrophilic solvent(s), or a combination thereof (i.e., one or more surfactant(s) and one or more hydrophilic solvent(s)), is used.

In preferred embodiments, omega-3 fatty acid oil is present in amounts of at least 30% w/w based on the total weight of the solvent system in the dosage form, more preferably at least 40%, even more preferably at least 50%, and most preferably at least 60%. In certain embodiments, the amount can be at least 70%, at least 80% or at least 90%.

The dosage form is stable at room temperature (about 23° C. to 27° C., preferably about 25° C.) and 60% relative humidity for a period of at least one month, preferably at least six months, more preferably at least one year, and most preferably at least two years. By “stable”, applicants mean that the solubilized statin does not precipitate out of solution to any appreciable degree, for example, in amounts of less than 10%, preferably less than 5%.

In addition, the dosage form preserves the statin from degradation. One embodiment of the invention includes a unit dosage form of a statin and omega-3 fatty acids in which at least 90% of the initial amount of statin in the dosage form at an initial measurement time (t₀) is maintained after one month storage at room temperature and 60% relative humidity.

The concentrated omega-3 fatty acids can be administered in a daily amount of from about 0.1 g to about 10 g, more preferably about 0.5 g to about 8 g, and most preferably from about 0.75 g to about 4 g. Preferably, in the unit dosage form, the omega-3 fatty acids are present in an amount from about 0.1 g to about 2 g, preferably about 0.5 g to about 1.5 g, more preferably about 1 g.

In one embodiment of the present invention, the statin can generally be present in an amount from about 2 mg to 80 mg, more preferably from about 5 mg to about 60 mg, and most preferably from about 10 mg to about 40 mg.

Pravastatin, which is known in the market as Pravachol® manufactured by Bristol-Myers Squibb, Princeton, N.J., is hydrophilic. Pravastatin is best absorbed without food, i.e., an empty stomach. The dosage of pravastatin in the combination product is preferably from 2.5 to 80 mg, preferably 5 to 60, and more preferably from 10 to 40 mg. In one variation, the combination product using pravastatin is taken at or around bedtime, e.g., 10 pm.

Lovastatin, which is marketed under the name Mevacor® by Merck, Whitehouse Station, N.J., is hydrophobic. Unlike pravastatin, lovastatin should be taken with meals and accordingly, in some embodiments, the combination product of concentrated omega-3 fatty acids and lovastatin should be taken with food. The dosage of lovastatin in the combination product is preferably from 2.5 to 100 mg, preferably 5 to 80 mg, and more preferably from 10 to 40 mg.

Simvastatin, which is marketed under the name Zocor® by Merck, Whitehouse Station, N.J., is hydrophobic. The dosage of simvastatin, in the combined administration of concentrated omega-3 fatty acids is preferably from 1 to 80 mg per day, preferably 2 to 60 mg, and more preferably from 5 to 40 mg.

Atorvastatin, which is marketed under the name Lipitor® by Pfizer, New York, N.Y., is hydrophobic and is known as a synthetic statin. The dosage of atorvastatin in the combination product is preferably from 2.5 to 100 mg, preferably 5 to 80 mg, and more preferably from 10 to 40 mg.

Fluvastatin, which is marketed under the name Lescol® by Novartis, East Hanover, N.J., is hydrophilic and is known as a synthetic statin. The dosage of fluvastatin in the combination product is preferably from 5 to 160 mg, preferably 10 to 120 mg, and more preferably from 20 to 80 mg.

Rosuvastatin is marketed under the name Crestor® by Astra Zeneca, Wilmington, Del. The dosage of rosuvastatin in the combination product is preferably from 1 to 80 mg, preferably 2 to 60 mg, and more preferably from 5 to 40 mg.

The most preferred form of statin according to the present invention is simvastatin, in a unit dosage in the combination product of 1 to 30 mg. Preferred daily dosages are 5 to 80 mg.

The daily dosages of statin and omega-3 fatty acids can be administered in from 1 to 10 dosages, with the preferred number of dosages from 1 to 4 times a day. The administration is preferably oral administration, although other forms of administration that provide a unit dosage of statin and omega-3 fatty acids may be used.

The present combination of a statin and omega-3 fatty acids may allow for a greater effect than any expected combined or additive effect of the two drugs alone. Moreover, the combined or additive effect of the two drugs may depend on the initial level of lipid parameter in the blood of a subject. For example, the triglyceride level of a subject is generally as normal if less than 150 mg/dL, borderline to high if within about 150-199 mg/dL, high if within about 200-499 mg/dL and very high if 500 mg/dL or higher. For any given lipid parameter, the present invention may be used to reduce the level of a “very high” down to a “high” or “borderline to high” in less than 48 weeks, preferably within 24 weeks, more preferably within 12 weeks, and most preferably within 6 weeks, 4 weeks or 2 weeks. The present invention may also be used to reduce the level of a “high” down to a “borderline to high” or “normal” in less than 48 weeks, preferably within 24 weeks, more preferably within 12 weeks, and most preferably within 6 weeks, 4 weeks or 2 weeks.

In some embodiments, the formulations of the present invention allow for improved effectiveness of each active ingredient, with one or both administered as a conventional full-strength dose, as compared to the formulations in the prior art. In other embodiments, the formulations of the present invention may allow for reduced dosages of statins and/or omega-3 fatty acids, as compared to the formulations in the prior art, while still maintaining or even improving upon the effectiveness of each active ingredient.

Any undesirable side effects may also be reduced as a result of the lower dosage amount and the reduction in excipients (e.g., surfactants).

The utilization of a combination product of a statin and omega-3 fatty acids overcomes the limitations of the prior art by improving the efficacy of statin and omega-3 fatty acids, and allows for a treatment with improved effectiveness and less excipients than in multiple administrations of omega-3 fatty acids and statins.

The combination product may be manufactured by any method known by those of ordinary skill in the art, by combining the statin(s) with the omega-3 fatty acid(s), and optionally with hydrophilic solvent(s) and/or surfactant(s) and/or other solubilizing agents and/or other excipients. A preferred method includes providing a hydrophilic solvent, such as ethanol, preferably in amounts of about 1 to 5% w/w based on the total weight of the solvent system in the dosage form, and adding thereto the statin(s), a surfactant, preferably in amounts of about 1 to 5% w/w based on the total weight of the solvent system in the dosage form, and the omega-3 fatty acid(s), preferably in amounts of about 10 to 50% w/w based on the total weight of the solvent system in the dosage form, to form a first mixture. The statin(s), surfactant and omega-3 fatty acids are preferably added to the hydrophilic solvent in sequential order, preferably while mixing. Thereafter, the first mixture is preferably mixed until the statin is dissolved in the first mixture. Thereafter, the remainder of the omega-3 fatty acid(s) is combined with the first mixture to form a second mixture.

### EXAMPLES

### Example 1

### Determining Solubility of Simvastatin in Omega-3 Fatty Acids

| | | |
|---|---|---|
| Component | Wt/Capsule | Wt |

Formulation 1

| | | |
|---|---|---|
| K85EE | 1000 mg | 50 g |
| Simvastatin | 1 mg | 0.05 g |

Mixed for 5 minutes. Clear solution observed.

Formulation 2

| | | |
|---|---|---|
| K85EE | 1000 mg | 50 g |
| Simvastatin | 2 mg | 0.1 g |

Mixed for 10 minutes. Clear solution observed.

Formulation 3

| | | |
|---|---|---|
| K85EE | 1000 mg | 50 g |
| Simvastatin | 4 mg | 0.2 g |

Mixed for 10 minutes. Clear solution observed.

Formulation 4

| | | |
|---|---|---|
| K85EE | 1000 mg | 50 g |
| Simvastatin | 6 mg | 0.3 g |

Mixed for 10 minutes. Clear solution observed.

Formulation 5

| | | |
|---|---|---|
| K85EE | 1000 mg | 50 g |
| Simvastatin | 8 mg | 0.4 g |

Mixed for 15 minutes. Clear solution observed.

Formulation 6

| | | |
|---|---|---|
| K85EE | 1000 mg | 50 g |
| Simvastatin | 9 mg | 0.45 g |

Mixed for 30 minutes. Turbid solution was observed. Kept aside for
precipitation. Precipitation was observed after one week.

### Example 2

### Pharmaceutical Compositions of Simvastatin and Omega-3 Acids

Formulation 1:
K85EE 1000 mg
Simvastatin 25 mg
2% Cremophor was added to dissolve the simvastatin. Mixed for 30 minutes.
Concentration of Cremophor was increased from 2% to 5% and then up to 7% while mixing for 30 minutes. Observed precipitation after 2 hours.

Formulation 2:
K85EE 1000 mg
Simvastatin 25 mg
2% Labrasol was added to dissolve the simvastatin. Mixed for 10 minutes.
Concentration of Labrasol was increased from 2% to 5% and mixed for 20 minutes. Observed precipitation after 2 hours.

Formulation 3:
K85EE 1000 mg
Simvastatin 25 mg
5% Tergitol NP-9 was added to dissolve the simvastatin. Mixed for 10 minutes.
Concentration of Tergitol was increased from 5% to 10% and mixed for 30 minutes. Observed precipitation after 2 hours.

Formulation 4:
K85EE 1000 mg
Simvastatin 25 mg
A combination of Tergitol NP-9 and Labrasol was added in the concentration of 10% Tergitol and 5% Labrasol. Mixed for 20 minutes. Clear solution was observed.

Formulation 5:
K85EE 1000 mg
Simvastatin 15 mg
Cremophor +Polysorbate 80 in 2:1 concentration
Labrasol 10%
10% Cremophor and 5% Polysorbate 80 was mixed in a beaker and simvastatin was added to it. Mixed well until all the drug was wetted then added 10% Labrasol. Mixed well. Oil was poured into the beaker and mixed for 15 minutes.
Turbid solution was observed.

Formulation 6:
K85EE 1000 mg
Simvastatin 15 mg
Capmul PG-8 10%
10% Capmul PG-8 was added to the simvastatin. Mixed well until all the drug was wetted then added the oil. Mixed well for 15 minutes. Clear solution was observed.

Formulation 7:
K85EE 1000 mg
Simvastatin 15 mg
Transcutol P 5%
5% Transcutol P was added to the simvastatin. Mixed well until all the drug was wetted then added the oil. Mixed well for 15 minutes. Clear solution was observed.

Formulation 8:
K85EE 1000 mg
Simvastatin 15 mg
Capmul PG-8 4%
Transcutol P 2%
Capmul PG-8 was added to the simvastatin. Mixed well until the drug was wetted. Added Transcutol P mixed well. Finally added oil and mixed for 15 minutes. Clear solution was observed.

Formulation 9:
K85EE 1000 mg
Simvastatin 20 mg
Capmul PG-8 12%
12% Capmul PG-8 was added to the simvastatin. Mixed well until all the drug was wetted then added the oil. Mixed well for 15 minutes. Clear solution was observed.

Formulation 10:
K85EE 1000 mg
Simvastatin 20 mg
Transcutol P 8%
8% Transcutol P was added to the simvastatin. Mixed well until all the drug was wetted then added the oil. Mixed well for 15 minutes. Clear solution was observed.

Formulation 11:
K85EE 1000 mg
Simvastatin 20 mg
Transcutol P 4%
Capmul PG-8 6%
Transcutol P was added to the simvastatin. Mixed well until the drug was wetted.
Added Capmul PG-8 mixed well. Finally added oil and mixed for 15 minutes.
Clear solution was observed.

Formulation 12:
K85EE 1000 mg
Simvastatin 15 mg
Capryol 90 2%
2% Capryol 90 was added to the simvastatin. Mixed well until the drug was wetted then oil was poured into the beaker slowly and mixed for 10 minutes.
Turbid solution was observed.

Formulation 13:
K85EE 1000 mg
Simvastatin 15 mg
Capryol 90 8%
2% Capryol 90 was added to the simvastatin. Mixed well until the drug was wetted then oil was poured into the beaker slowly and mixed for 10 minutes. 6% more of Capryol 90 was added and mixed well. Clear solution was observed.

Formulation 14:
K85EE 1000 mg
Simvastatin 15 mg
Lauro Glycol 90 5%
5% Lauro Glycol 90 was added to the simvastatin. Mixed well until the drug was wetted then oil was poured into the beaker slowly and mixed for 10 minutes.
Turbid solution was observed.

Formulation 15:
K85EE 1000 mg
Simvastatin 15 mg
Lauro Glycol 90 12%
5% Lauro Glycol 90 was added to the simvastatin. Mixed well until the drug was wetted then oil was poured into the beaker slowly and mixed for 10 minutes. 7% more of Lauro Glycol 90 was added and mixed well. Clear solution was observed.

Formulation 16:
K85EE 1000 mg
Simvastatin 15 mg
Labrafil M 1944CS 15%
15% Labrafil was added to the simvastatin. Mixed well until the drug was wetted then oil was poured into the beaker slowly and mixed for 10 minutes. Turbid solution was observed.

Formulation 17:
K85EE 1000 mg
Simvastatin 15 mg
Plurol Oleique CC497 12%
Oil Component -1000 mg
12% Plurol Oleique was added to the simvastatin. Mixed well until the drug was wetted then oil was poured into the beaker slowly and mixed for 10 minutes.
Turbid solution was observed.

Formulation 18:
K85EE 1000 mg
Simvastatin 15 mg
Capryol 90 5%
Lauro Glycol 90 5%
5% Capryol 90 was added to the simvastatin. Mixed well until the drug was wetted then Lauro Glycol was added and mixed well. Oil was poured into the beaker slowly and mixed for 10 minutes. Clear solution was observed.

Formulation 19:
K85EE 1000 mg
Simvastatin 15 mg
Capryol 90 5%
Capmul PG-8 5%
5% Capryol 90 was added to the simvastatin. Mixed well until the drug was wetted then Capmul PG-8 was added and mixed well. Oil was poured into the beaker slowly and mixed for 10 minutes. Clear solution was observed.

Formulation 20:
K85EE 1000 mg
Simvastatin 15 mg
Capryol 90 5%
Cremophor 5%
5% Capryol 90 was added to the simvastatin. Mixed well until the drug was wetted then Cremophor was added and mixed well. Oil was poured into the beaker slowly and mixed for 10 minutes. Slightly turbid solution was observed.

Formulation 21:
K85EE 1000 mg
Simvastatin 15 mg
Capryol 90 5%
Capmul PG-8 2%
5% Capryol 90 was added to the simvastatin. Mixed well until the drug was wetted then 2% Capmul PG-8 was added and mixed well. Oil was poured into the beaker slowly and mixed for 10 minutes. Slightly turbid solution was observed.

### Example 3

### Unit Dose Formulations of Statin and Omega-3 Acids

Formulation 1: gelatin capsule
K85EE 1000 mg
Simvastatin 20 mg
Dehydrated alcohol 39.5 mg
Capmul MCM 20 mg

Formulation 2: gelatin capsule
EPAX7010EE 750 mg
Atorvastatin 10 mg
Polyethylene glycol 17.5 mg
Tergitol NP-9 10 mg

Formulation 3: gelatin capsule
TG2162 850 mg
Fluvastatin 30 mg
Propylene glycol 15 mg
Capryol 90 15 mg

All references cited herein are incorporated by reference herein in their entirety.

We claim:

1. A pharmaceutical composition in unit dose form, comprising an essentially homogeneous solution comprising a statin essentially dissolved in solvent system comprising natural or synthetic omega-3 fatty acids or pharmaceutically acceptable esters, derivatives, conjugates, precursors or salts thereof, or mixtures thereof, wherein less than 10% of the statin is undissolved in the solvent system.

2. The pharmaceutical composition of claim 1, wherein the statin and the omega-3 fatty acids are the only active agents in the pharmaceutical composition.

3. The pharmaceutical composition of claim 1, wherein the solvent system further comprises at least one solubilizer in an amount of 50% or less w/w based on the total weight of the solvent system.

4. The pharmaceutical composition of claim 3, wherein the at least one solubilizer comprises a hydrophilic solvent.

5. The pharmaceutical composition of claim 4, wherein the hydrophilic solvent is present in an amount of 20% or less w/w based on the total weight of the solvent system.

6. The pharmaceutical composition of claim 3, wherein the at least one solubilizer comprises a surfactant.

7. The pharmaceutical composition of claim 6, wherein the surfactant is present in an amount of 20% or less w/w based on the total weight of the solvent system.

8. The pharmaceutical composition of claim 3, wherein the at least one solubilizer comprises a hydrophilic solvent and a surfactant.

9. The pharmaceutical composition of claim 8, wherein the hydrophilic solvent and the surfactant are present together in an amount of 20% or less w/w based on the total weight of the solvent system.

10. The pharmaceutical composition of claim 1, wherein no more than 10% of the dissolved statin precipitates out of the solution when the pharmaceutical composition is stored at room temperature and 60% relative humidity for a period of at least one month.

11. The pharmaceutical composition of claim 1, wherein the statin is simvastatin.

12. A pharmaceutical composition in unit dose form, comprising a statin and natural or synthetic omega-3 fatty acids or pharmaceutically acceptable esters, derivatives, conjugates, precursors or salts thereof, or mixtures thereof, wherein at least 90% of the initial amount of the statin in the composition at an initial measurement time (t₀) is maintained after one month storage at room temperature and 60% relative humidity.

13. A method of treating a subject having one or more conditions selected from the group consisting of hypertriglyceridemia, hypercholesteremia, coronary heart disease (CHD), heart failure, cardiac arrhythmias, ischemic dementia, hypertension, coagulation related disorders, nephropathy, retinopathy, cognitive disorders, autoimmune diseases, inflammatory diseases, metabolic syndrome, vascular disease, atherosclerotic disease and related conditions, comprising administering to the subject a pharmaceutical composition as claimed in claim 1.

14. The method of claim 13, wherein prior to the first administration to the subject of the pharmaceutical composition, the subject has triglyceride levels of about 200 to about 499 mg/dl.

15. The method of claim 13, wherein prior to the first administration to the subject of the pharmaceutical composition, the subject has triglyceride levels of greater than 499 mg/dl.

16. The method of claim 13, wherein prior to the first administration to the subject of the pharmaceutical composition, the subject has triglyceride levels of about 150 to about 199 mg/dl.

17. A method for the treatment and/or prevention and/or reduction of cardiac events and/or cardiovascular events and/or vascular events and/or symptoms in a subject, comprising administering to the subject a pharmaceutical composition as claimed in claim 1.

18. The method of claim 17, wherein prior to the first administration to the subject of the pharmaceutical composition, the subject has triglyceride levels of about 200 to about 499 mg/dl.

19. The method of claim 17, wherein prior to the first administration to the subject of the pharmaceutical composition, the subject has triglyceride levels of greater than 499 mg/dl.

20. The method of claim 17, wherein prior to the first administration to the subject of the pharmaceutical composition, the subject has triglyceride levels of about 150 to about 199 mg/dl.
